(19) United States
(12) Patent Application Publication
(10) Pub. No.: US 2020/0246298 A1
(43) Pub. Date: Aug. 6, 2020

(54) Composition for Preventing or Treating Asthma Comprising Fatty Acid as Active Ingredient

(71) Applicant: Dongshin University Industry-Academy Cooperation, Naju-si (KR)

(72) Inventors: Dae Hun Park, Daejeon (KR); Deuk Sil Oh, Gwangju (KR); Seung Sik Cho, Gwangju (KR)

(73) Assignee: Dongshin University Industry-Academy Cooperation, Naju-si (KR)

(21) Appl. No.: 16/276,643

(22) Filed: Feb. 15, 2019

(30) Foreign Application Priority Data
Jan. 31, 2019 (KR) 10-2019-0013179

(51) Int. Cl. A61K 31/20; A61P 11/06

(52) U.S. Cl. CPC A61K 31/20; A61P 11/06

(57) ABSTRACT

The present invention relates to a composition for preventing or treating asthma, having, an active ingredient, a saturated or unsaturated fatty acid having 12 to 22 carbon atoms. The saturated or unsaturated fatty acid functions to inhibit or eliminate various asthma symptoms induced by ovalbumin. Specifically, the fatty acid inhibits the proliferation of leukocytes, neutrophils, lymphocytes and monocytes in BALF and serum, inhibits the proliferation of bronchial epithelial cells, reduces mucus in bronchioles, and reduces infiltration of inflammatory cells around bronchioles and blood vessels in a dose-dependent manner. Also, the fatty acid induces a decrease in the expression of IFN-γ, IL-12p40, IL-4, IL-5, IL-13 and TNF-α, which are Th2 cell-related cytokines. Therefore, the fatty acid overcomes the side effects of current asthma therapeutic agents, has an excellent therapeutic effect without having toxicity, and may advantageously be used as a composition for preventing, treating or alleviating asthma.

### CROSS REFERENCE TO RELATED APPLICATION

The present application claims the benefit of priority od Korean Patent Application No. KR 10-2019-0013179 filed Jan. 31, 2019, the contents of which are incorporated herein by reference.

### BACKGROUND OF THE INVENTION

### Field of the Invention

The present invention relates to a composition for preventing or treating asthma, comprising a fatty acid as an active ingredient, and more particularly to a pharmaceutical composition for preventing or treating asthma or a food composition for preventing or alleviating asthma, which comprises, as an active ingredient, a saturated or unsaturated fatty acid having 12 to 22 carbon atoms or a pharmaceutically acceptable salt thereof.

### Description of the Prior Art

The World Health Organization (WHO) reported that there were 383,000 deaths due to asthma in 2015 and that asthma affects 235 million people worldwide and is common, particularly among young people and the elderly. Asthma is pulmonary hyperresponsiveness, a type I allergy, and its symptoms vary widely, from apnea to death (National Asthma Education and Prevention Program, N.A.E.a.P., 2002; Kay 2001). It is impossible to completely cure asthma, which is a major health problem (Slejko et al., 2014). There are many types of allergens in the environment, including indoor allergens, such as house dust mites, pet dander, etc., and outdoor allergens, such as air pollutants, chemicals, tobacco smoke, etc. (Plattes-Mills et al., 1997; Burge and Rogers, 2000).

Repeated uptake of allergens may disturb the immune system, especially the Th type 1 (Th1)/Th2 system, and may cause the onset of asthma (National Asthma Education and Prevention Program, N.A.P, 2002, Kay 2001). Interferon-gamma (IFN-γ) and interleukin 12 (IL-12) are Th1-related cytokines, TH2 cells secrete some cytokines, such as IL-4, IL-5, IL-13 and the like, and Th17 cells are important in regulating the Th1/Th2 balance that controls the onset of asthma (Mosmann and Coffman, 1989; Singh et al., 2014). The levels of Th2-related cytokines, such as IL-4, IL-5, and IL-13, in most asthma patients, are very high (Antczak et al., 2016; Foster et al., 1996; van der Pouw Kraan et al., 1998; Wenzel et al., 2007), and Th2-related cytokines are regulated by GATA-3 and Th2 cell transcription factors (Yagi et al., 2011). Th1-related cytokines, such as IFN-γ and IL-12p40, down-regulate the levels of Th1-related cytokines, and T-bet is a Th1 cell transcription factor (Szabo et al., 2000; Zhu et al., 2012).

In order to treat asthma, inhaled combination therapy has recently been used, which usually consists of corticosteroids and long-acting β₂-agonist or leukotriene modifiers (Mishra et al., 2013). In asthma treatment, the most difficult things are that it is hard to perfectly cure, and that general medications have a lot of adverse effects such as growth suppression, eye problem, hypertension, hyperlipidemia, gastric ulcer, neurotoxicity, and so on (Wise 2014 Ciriaco et al., 2013). Efforts to develop safe and effective anti-asthmatic drugs from natural products have increased in recent years (Bang et al., 2015; Lee et al., 2017; Seo et al., 2016). For example, Korean Patent No. 10-1917504 discloses a composition for preventing or treating asthma, which comprises an extract of Aplysia kurodai, and Korean Patent No. 10-1839721 discloses a pharmaceutical composition for preventing or treating an allergic disease, such as asthma or atopy, which comprises baicalein as an active ingredient.

Meanwhile, fatty acids are components of in vivo cell membranes, which can be supplied from a variety of vegetable and animal edible oils and fats. Studies on fatty acids have been focused on the physiological activities of essential fatty acids, such as linoleic acid, linoleic acid, long-chain polyunsaturated fatty acids or the like, which are difficult to synthesize in vivo. These essential fatty acids, including long-chain polyunsaturated fatty acids, have been reported to have various physiological activities, such as learning ability improvement, cholesterol reduction, and cancer cell suppression. However, there is no report yet on the anti-asthmatic effect of fatty acids.

Accordingly, the present inventors have conducted continued studies to develop a new substance having the effect of preventing or treating asthma, and as a result, have newly found that oleic acid and palmitic acid, which are fatty acids, inhibit the proliferation of leukocytes, neutrophils, lymphocytes and monocytes in BALF and serum and exhibit an anti-asthmatic effect by inducing a decrease in the expression of IFN-γ, IL-12p40, IL-4, IL-5, IL-13 and TNF-α, which are Th2 cell-related cytokines, thereby completing the present invention.

### SUMMARY OF THE INVENTION

Therefore, it is an object of the present invention to provide a pharmaceutical composition for preventing or treating asthma, which comprises, as an active ingredient, a fatty acid having an anti-asthmatic effect.

Another object of the present invention is to provide a food composition for preventing or alleviating asthma, which comprises, as an active ingredient, a fatty acid having an anti-asthmatic effect.

Another object of the present invention is to provide a a method for preventing or treating asthma.

To achieve the above objects, the present invention provides a pharmaceutical composition for preventing or treating asthma, which comprises, as an active ingredient, a fatty acid having 12 to 22 carbon atoms or a pharmaceutically acceptable salt thereof.

The present invention also provides a food composition for preventing or alleviating asthma, which comprises, as an active ingredient, a fatty acid having 12 to 22 carbon atoms or a nutritionally acceptable salt thereof.

Further, the present invention also provides a method for preventing or treating asthma, comprising a step of administering said pharmaceutical composition to a subject suspected of having asthma.

In the present invention, the fatty acid may be a saturated or unsaturated fatty acid having 12 to 22 carbon atoms, preferably a saturated or unsaturated fatty acid having 14 to 20 carbon atoms, more preferably a saturated or unsaturated fatty acid having 16 to 18 carbon atoms.

The saturated fatty acid may be any one or a mixture of two or more selected from the group consisting of lauric acid, myristic acid, palmitic acid, and stearic acid. Preferably, it may be palmitic acid or stearic acid.

The unsaturated fatty acid may be any one or a mixture of two or more selected from the group consisting of oleic acid, linoleic acid, and linolenic acid. Preferably, it may be oleic acid.

In the present invention, fatty acids may be used in the form of pharmaceutically/nutritionally acceptable salts, and the salts are preferably acid addition salts formed by pharmaceutically/nutritionally acceptable free acids. The acid addition salts are obtained from inorganic acids, such as hydrochloric acid, nitric acid, phosphoric acid, sulfuric acid, hydrobromic acid, hydroiodic acid, nitrous acid or phosphorous acid, or nontoxic organic acids, such as aliphatic mono- and di-carboxylate, phenyl-substituted alkanoate, hydroxy alkanoate and alkanedioate, aromatic acids, aliphatic and aromatic sulfonic acids. Examples of these pharmaceutically/nutritionally nontoxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, hydroxybutyrate, glycolate, malate, tartrate, methanesulfonate, propanesulfonate, naphthalene-1-sulfonate, naphthalene-2-sulfonate or mandelate.

The acid addition salt according to the present invention may be prepared by a conventional method. For example, it may be prepared by dissolving the fatty acid in an excessive amount of an acid aqueous solution and precipitating the formed salt using a water-miscible organic solvent, for example, methanol, ethanol, acetone or acetonitrile. Alternatively, it may also be prepared either by evaporating the solvent or an excess of the acid, followed by drying, or by suction-filtering the precipitated salt.

Alternatively, a pharmaceutically acceptable metal salt may be prepared using a base. An alkali or alkaline metal salt is obtained, for example, by dissolving a compound in an excessive amount of an alkali metal hydroxide or alkaline earth metal hydroxide solution, filtering a non-dissolved compound acid salt, and evaporating and drying the filtrate. In this case, it is pharmaceutically suitable to prepare a sodium, potassium or calcium salt as the metal salt. In addition, a silver salt corresponding to the metal salt is obtained by reacting an alkali metal or alkaline earth metal salt with a suitable silver salt (e.g., silver nitrate).

As used herein, the term “asthma” is a condition in which the bronchi in the lungs are very sensitive. Specifically, asthma is a disease that sometimes causes symptoms, including bronchial narrowing, wheezing, and severe cough, and is an allergic disease caused by the allergic inflammatory reaction of the bronchi. Typical symptoms of asthma include dyspnea, cough, wheezing and the like. Therapeutic agents that are typically used to treat asthma include symptom relievers (bronchodilators) that dilate narrowed bronchi within a short time, or disease control agents (anti-inflammatory agents and leukotriene modulators) that prevents asthma attack by inhibiting bronchial allergic inflammation. In the present invention, the asthma may be bronchial asthma, allergic asthma, atopic asthma, non-atopic asthma, exercise-induced asthma, aspirin asthma, cardiogenic asthma, or alveolar asthma, but is not limited thereto.

The composition of the present invention may contain a fatty acid having 12 to 22 carbon atoms or a pharmaceutically/nutritionally acceptable salt thereof in an amount of 0.005 to 50 wt %, more preferably 0.01 to 30 wt %, most preferably 0.1 to 10 wt %, based on the total weight of the composition. If the content of the fatty acid having 12 to 22 carbon atoms or a pharmaceutically/nutritionally acceptable salt thereof is less than 0.005 wt %, an anti-asthmatic effect which is the effect of the present invention cannot be obtained, and if the content is more than 50 wt %, problems arise in that inefficiency may occur because the effect does not increase in proportion to an increase in the content and in that the stability of formulation is not ensured.

It was found in the present invention that the fatty acid having 12 to 22 carbon atoms according to the present invention reduced the number of inflammatory cells around bronchi or blood vessels or reduced the secretion of mucus from bronchial epithelial goblet cells. Bronchoalveolar lavage was performed in asthma patients and airway inflammation was examined, and as a result, it was shown that the number of lymphocytes, mast cells, eosinophils and activated macrophages in the bronchoalveolar lavage fluid increased. It is generally known that asthma is an airway inflammatory disease and that various inflammatory cells are mostly activated to secrete various mediator substances to induce asthma, indicating that reduction of inflammatory cells is associated with the treatment of asthma (Haley K J, et al., Am J Respir Crit Care Med, 1998; 158: 565-72). Meanwhile, in asthma, in addition to airway narrowing and the infiltration of inflammatory cells into bronchi, goblet cells are formed to secrete mucus, and collagen deposition is prominent. Thus, an increase in bronchial mucus secretion is also known as a kind of asthma symptom.

In one example of the present invention, inflammation in lung tissue was observed by H&E (Hematoxylin & Eosin) staining, and bronchial mucus secretion was observed by PAS (periodic acid Schiff) staining. As a result, i) it was shown that extensive infiltration of inflammatory cells around the bronchi and blood vessels of an asthma-induced group was observed, whereas infiltration of inflammatory cells around the bronchi and blood vessels in all groups administered with a fatty acid having 12 to 22 carbon atoms decreased, and ii) it was observed that an increase in mucus secretion from bronchial epithelial goblet cells in the asthma-induced group increased, whereas mucus secretion from bronchial epithelial goblet cells in all the groups administered with a fatty acid having 12 to 22 carbon atoms significantly decreased. These results also indicate that the fatty acid having 12 to 22 carbon atoms, for example, oleic acid, stearic acid or palmitic acid, is effective for the treatment of asthma.

As used herein, the term “preventing” means all actions that inhibit or delay the onset of asthma by administering the composition, and the term “treating” all actions that alleviate or beneficially change asthma symptoms by administering the composition.

The pharmaceutical composition comprising the fatty acid having 12 to 22 carbon atoms according to the present invention may additionally comprise pharmaceutically acceptable carriers, excipients or diluents which are commonly used in the preparation of pharmaceutical compositions.

The pharmaceutical composition may have any one formulation selected from the group consisting of tablet, pill, powder, granule, capsule, suspension, solution, emulsion, syrup, sterilized aqueous solution, non-aqueous solution, freeze-drying formulation, and suppository, and may be formulated in various forms for oral or parenteral administration. For formulation, the composition is formulated using diluents or excipients, such as fillers, extenders, binders, wetting agents, disintegrants, surfactants, etc., which are commonly used. Solid formulations for oral administration include tablet, pill, powder, granule, capsule and the like, and such solid formulations comprise, in addition to one or more active ingredients, at least one excipient, for example, starch, calcium carbonate, sucrose, lactose or gelatin. In addition to simple excipients, lubricants such as magnesium stearate or talc may also be used. Liquid formulations for oral administration include suspension, solution, emulsion, and syrup, and may comprise various excipients, for example, a wetting agent, a flavoring agent, an aromatic and a preservative, in addition to simple diluents that are frequently used, such as water and liquid paraffin. Formulations for parenteral administration include sterilized aqueous solution, non-aqueous solution, suspension, emulsion, freeze-dried formulation, and suppository. As a non-aqueous solvent or a suspending agent, propylene glycol, polyethylene glycol, vegetable oil such as olive oil, injectable ester such as ethyl oleate, or the like, may be used.

The pharmaceutical composition of the present invention may be administered in a pharmaceutically effective amount. As used herein, the term “pharmaceutically effective amount” refers to an amount sufficient to treat diseases, at a reasonable benefit/risk ratio applicable to any medical treatment. The effective dosage level may be determined depending on various factors, including the subject's type, the disease severity, the subject's age and sex, the type of disease, the activity of the drug, sensitivity to the drug, the time of administration, the route of administration, excretion rate, the duration of treatment, drugs used in combination with the composition, and other factors well known in the medical field. The pharmaceutical composition of the present invention may be administered individually or in combination with other therapeutic agents, and may be administered sequentially or simultaneously with conventional therapeutic agents. In addition, the composition can be administered in a single or multiple dosage form. It is important to administer the composition in the minimum amount that can exhibit the maximum effect without causing side effects, in view of all the above-described factors, and this amount can be easily determined by a person skilled in the art. The preferred dose of the composition of the present invention may vary depending on the patient's conditions and weight, the severity of the disease, the type of drug, the route of administration and the duration of treatment. The suitable total daily dose may be determined by an attending physician within the scope of sound medical judgment, but the composition may generally be administered once or several times at a dose of 0.001 to 1000 mg/kg, preferably 0.05 to 200 mg/kg, more preferably 0.1 to 100 mg/kg. The composition may be applied to any subject for the purpose of preventing or treating asthma. For example, the composition of the present invention may be applied to any subjects, including non-human animals, such as monkeys, dogs, cats, rabbits, marmots, rats, mice, cattle, sheep, pigs and goats, humans, birds, and fish, and modes of administration of the composition include any conventional methods known in the art. For example, the composition may be administered orally or by intravenous, intramuscular or subcutaneous injection.

In another aspect, the present invention provides a food composition for alleviating or preventing asthma, which comprises, as an active ingredient, a fatty acid having 12 to 22 carbon atoms or a nutritionally acceptable salt thereof.

Specifically, the fatty acid having 12 to 22 carbon atoms or nutritionally acceptable salt thereof according to the present invention may be contained in a food composition for the purpose of alleviating or preventing asthma. The asthma is as described above. As used herein, the term “alleviating” refers to all actions that alleviate or beneficially change asthma symptoms in a subject suspected or diagnosed of having asthma by administering the composition.

When the composition of the present invention is contained in a food, the composition may be added as it is or may be used in combination with other health functional food or health functional food ingredients, and may be appropriately used according to a conventional method. The content of the active ingredient may be suitably determined according to the intended use. Generally, in the preparation of a food or a beverage, the composition of the present invention may preferably be added in an amount of 15 parts by weight or less, more preferably 10 parts by weight, based on the total weight of the food or the beverage. However, in the case of long-term intake for the purpose of health control and hygiene, the content may be lower than the lower limit of the above range, and since there is no problem in terms of stability, the active ingredient may also be used in an amount exceeding the upper limit of the above range.

There is no particular limit to the kind of food that may contain the composition of the present invention. Specific examples of foods to which the composition of the present invention may be added include meats, sausages, bread, chocolate, candies, snack, confectionery, pizza, noodles, gum, dairy products including ice cream, various soups, beverages, teas, drinks, alcoholic beverages and multi-vitamin preparations. The foods include all health foods in a conventional sense, and also include foods which are used as animal feeds. In addition, when the food composition of the present invention is to be used as a beverage, it may additionally contain various sweetening agents or natural carbohydrates as in conventional beverages. The natural carbohydrates include monosaccharides, such as glucose and fructose, disaccharides, such as maltose and sucrose, polysaccharides, such as dextrin and cyclodextrin, and sugar alcohols, such as xylitol, sorbitol and erythritol. The content of the natural carbohydrate may preferably be 0.01 to 0.04 g, more preferably 0.02 to 0.03 g, based on 100 ml of the composition of the present invention. Examples of the sweetener include natural sweeteners, such as thaumatin and stevia extract, and synthetic sweeteners, such as saccharin and aspartame. In addition, the food composition of the present invention may further contain various nutritional supplements, vitamins, electrolytes, flavoring agents, coloring agents, pectic acid and its salt, alginic acid and its salt, organic acids, protective colloids, thickeners, pH adjusters, stabilizers, preservatives, glycerin, alcohols, carbonizing agents that are used in carbonated beverages, etc. In addition, the food composition may contain fruit fresh for preparation of natural fruit juice beverages, fruit juice beverages and vegetable juices.

In still another aspect, the present invention provides a method for preventing or treating asthma, comprising a step of administering the pharmaceutical composition of the present invention to a subject suspected of having asthma. In the present invention, the subject suspected of having asthma means all animals, including humans, who have developed asthma or are at risk of developing asthma, and asthma may be efficiently treated by administering, to the subject suspected of having asthma, the pharmaceutical composition comprising the compound or pharmaceutically acceptable salt thereof according to the present invention. The asthma is as described above.

As used herein, the term “administering” means introducing the pharmaceutical composition of the present invention into a subject suspected of having asthma by any suitable method. The composition of the present invention may be administered by various routes, including oral or parenteral routes, as long as it can reach a target tissue. The treating method according to the present invention may comprise administering a pharmaceutically effective amount of the pharmaceutical composition comprising the fatty acid having 12 to 22 carbon atoms or a pharmaceutically acceptable salt thereof. The suitable total daily dose of the pharmaceutical composition may be determined by an attending physician within the scope of sound medical judgment, and the composition may generally be administered once or several times at a dose of 0.001 to 1000 mg/kg, preferably 0.05 to 200 mg/kg, more preferably 0.1 to 100 mg/kg.

However, for the purpose of the present invention, the specific therapeutically amount of the pharmaceutical composition for any particular patient preferably varies depending on various factors, including the kind and degree of response to be achieved, specific compositions according to whether or not other agents are used therewith, the patient's age, body weight, health conditions, sex and diet, the time and route of administration, the secretion rate of the composition, the duration of treatment, the drug(s) administered in combination or simultaneously with the specific composition, and similar factors well known in the medical field.

### BRIEF DESCRIPTION OF THE DRAWINGS

FIG. 1 shows the results measuring a differential cell count by a blood cell analysis in the BALF of groups administered with oleic acid.

FIG. 2 shows the change in the number of WBC cells in an OVA-sensitized group by administration of oleic acid.

FIG. 3 shows the change in the number of eosinophils (EOs) in an OVA-sensitized group by administration of oleic acid.

FIG. 4 shows the change in the number of neutrophils (NEs) in an OVA-sensitized group by administration of oleic acid.

FIG. 5 shows the change in the number of lymphocytes (LYs) in an OVA-sensitized group by administration of oleic acid.

FIG. 6 shows the change in the number of monocytes (MOs) in an OVA-sensitized group by administration of oleic acid.

FIG. 7 shows the results measuring a differential cell count by a blood cell analysis in the BALF of groups administered with palmitic acid.

FIG. 8 shows the change in the number of WBC cells in an OVA-sensitized group by administration of palmitic acid.

FIG. 9 shows the change in the number of eosinophils (EOs) in an OVA-sensitized group by administration of palmitic acid.

FIG. 10 shows the change in the number of neutrophils (NEs) in an OVA-sensitized group by administration of palmitic acid.

FIG. 11 shows the change in the number of lymphocytes (LYs) in an OVA-sensitized group by administration of palmitic acid.

FIG. 12 shows the change in the number of monocytes (MOs) in an OVA-sensitized group by administration of palmitic acid.

FIG. 13 shows the change in IgE in an OVA-sensitized group by administration of oleic acid.

FIG. 14 shows the change in IgE in an OVA-sensitized group by administration of palmitic acid.

FIG. 15 shows the results of H&E staining for an OVA-sensitized group and groups administered with oleic acid.

FIG. 16 shows the results of H&E staining for an OVA-sensitized group and groups administered with palmitic acid.

FIG. 17 shows the results of PAS staining for an OVA-sensitized group and groups administered with oleic acid.

FIG. 18 shows the results of PAS staining for an OVA-sensitized group and groups administered with palmitic acid.

FIGS. 19A, 19B, 19C, and 19D show the changes in activities of the Th1 cell transcription factor T-bet and the Th2 cell transcription factor GATA-3 by administration of palmitic acid.

FIG. 20 shows the change in expression level of IFN-γ in an OVA-sensitized group by administration of palmitic acid.

FIG. 21 shows the change in expression level of IL-12p40 in an OVA-sensitized group by administration of palmitic acid.

FIG. 22 shows the change in expression level of IL-4 in an OVA-sensitized group by administration of palmitic acid.

FIG. 23 shows the change in expression level of IL-5 in an OVA-sensitized group by administration of palmitic acid.

FIG. 24 shows the change in expression level of IL-13 in OVA-sensitized groups by administration of palmitic acid.

FIG. 25 shows the change in expression level of TNF-α in an OVA-sensitized group by administration of palmitic acid.

FIG. 26 shows the change in expression level of IL-6 in an OVA-sensitized group by administration of palmitic acid.

FIG. 27A-27G shows the changes in helper T cell-related cytokines by administration of palmitic acid.

### DETAILED DESCRIPTION OF THE INVENTION

Hereinafter, the present invention will be described in detail with reference to examples in order to facilitate understanding of the present invention. However, the examples according to the present invention can be modified in various other forms, and the scope of the present invention should not be construed as being limited to these examples. The examples of the invention are provided to more fully explain the present invention to those skilled in the art.

### Materials and Methods

This experiment was performed in order to examine the anti-asthmatic effects of fatty acids after inducing asthma in experimental animals by ovalbumin (OVA).

### (1) Experimental Animals

Experimental animals used in the experiment were chosen because of the accumulated abundant test data and their ease of analysis and evaluation of experimental results. BALB/c mice, which are specific pathogen member (SPF) mice, were chosen based on the related literature. Experimental animals were provided in the minimum number so as to be able to analyze experimental results and were divided into a total of 9 groups according to the substance to be administered, and each group consisted of 8 animals.

### (2) Experimental Method

For an animal experimental schedule, purchased animals were observed at least once a day during their acclimation in an animal experimental facility in which the experiment was performed. In test groups, the animals determined to be healthy during the acclimation period were ranked by measuring the weight, and were randomly grouped so that the average weight of each group was uniform. The animals were divided into a total of 9 groups: a control group; a control group with OVA-induced asthma; a positive control group; groups administered with each of 50 mg/kg, 125 mg/kg and 250 mg/kg of oleic acid; and groups administered with each of 5 mg/kg, 25 mg/kg and 50 mg/kg of palmitic acid. The animals were sensitized via intraperitoneal injection of OVA once a week for 2 weeks after the acclimation period. During 5 days of week 3, dexamethasone (DEX) and the test substances oleic acid and palmitic acid were orally administered in the morning and 5% OVA solution was inhaled in the afternoon. The test substance was orally administered in an amount of 28 μL per weight (g) of the experimental animal, and OVA for sensitization was administered in a constant amount regardless of the weight. After 5 days of administration, autopsy was performed the next day.

### (3) Administration Method

Intraperitoneal administration: based on the related literature, 20 μg OVA+1 mg aluminum hydroxide+500 μL/head of normal saline were administered. For a total of 50 animals to be administered, 1000 μg OVA+50 mg aluminum hydroxide+25 mL of normal saline were prepared. The animals were sensitized by intraperitoneal injection once a week for 2 weeks, and injected with the test substance by a 1 mL syringe.

Oral administration: the control group and the asthma-induced control group were administered with saline, and oleic acid and palmitic acid were used in dissolved in saline at various concentrations. Dexamethasone was dissolved in saline by stirring and prepared according to a final dose. All preparations were prepared on the day of administration.

Inhalation: a solution containing 1 g OVA+20 mL normal saline (5% OVA solution) was prepared, stirred and administered. A forced inhalation was performed using a nebulizer (OMRON, NE-U17) for 5 days, and systemic exposure was performed using an inhalation exposure chamber.

### (4) Experimental Analysis Method

Collection of bronchoalveolar lavage fluid (BALF): the anesthetized animal tissue was opened with tweezers to secure the trachea, and then a surgical thread was placed underneath and loosely tied, and the trachea was slightly cut with scissors. Then, 0.2 mL of PBS was placed into a syringe with the needle removed, and the syringe was inserted into a sonde and then injected into the trachea. After injection, BALF was collected by injecting PBS again. 0.2 mL of BALF was collected three times in the same manner. The first BALF was placed in tube No. 1, the second and third BALFs were placed in tube No. 2.

Storage of tissue: ½ of the autopsy tissue was frozen quickly in liquid nitrogen, stored in an ultralow-temperature freezer or liquid nitrogen container (about −75 ° C.), and used for PCR/Western blotting, and the remaining ½ was stored in 10% formalin solution and used for histopathology and immunohistochemistry (IHC).

### EXPERIMENTAL EXAMPLE 1

### Sample Analysis

### (1) Blood Cell Analysis in BALF

The blood cell analysis in BALF were performed (Lee et al., 2017; Bang et al., 2015). One day after the final treatment, the mice were anesthetized with intraperitoneal injections of 50 mg/kg Zoletin (Virbac, Fort Worth, Tex., USA), and their tracheas were cannulated with disposable animal feeding needles. Lavages were performed with three 0.4 ml aliquots of cold phosphate-buffered saline (PBS). The BALF samples were collected and immediately centrifuged at 3000 rpm for 5 minutes at 4° C. (Sorvall Legend Micro 17R, Thermo Fisher Scientific, Inc. Marietta, Ohio, USA). The cell pellets were resuspended in PBS for total and differential cell counts. The number of total cells and differential cells were counted by the Hemavet Multispecies Hematology System (Drew Scientific Inc., Waterbury, Conn., USA). Some resuspended cell pellets were stained by the Diff-Quick method (Skipper and DeStephano, 1989). The number of total cells was counted using a hemocytometer, and the number of eosinophils in BALF was counted on cytospin preparations that were stained using the Kwik-Diff staining set (Thermo Fisher Scientific Inc., Pittsburgh, Pa., USA).

### (2) Results of BALF and Serum Analysis in Groups Administered with Oleic Acid

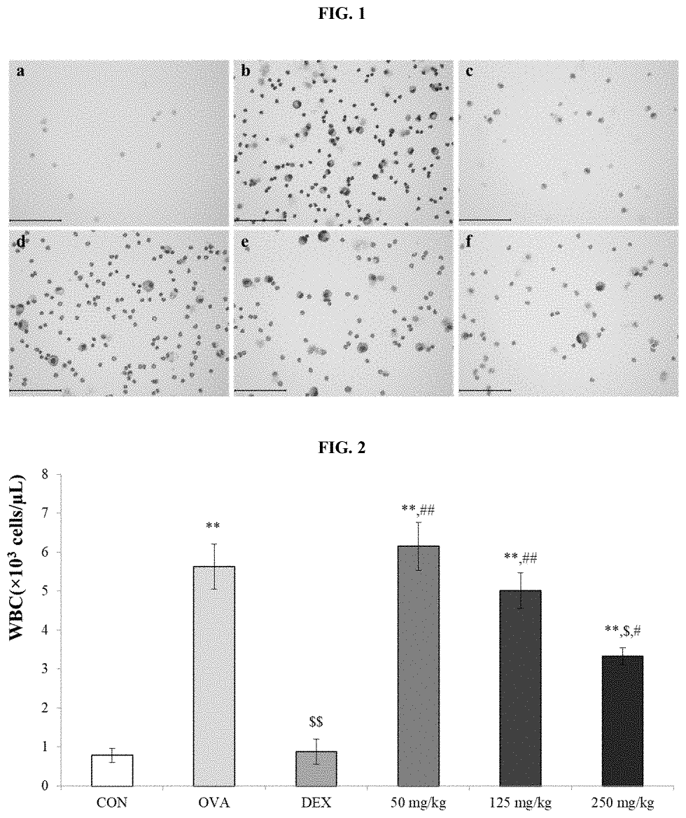

FIG. 1 shows the results measuring a differential cell count by a blood cell analysis in the BALF of the groups administered with oleic acid (a: CON, b: OVA, c: DEX, d: 50 mg/kg, e: 125 mg/kg, f: 250 mg/kg). As can be seen therein, the number of differential cells dose-dependently decreased in the groups administered with oleic acid.

FIG. 2 shows the change in the number of WBC cells in the OVA-sensitized group by administration of oleic acid (a: CON, b: OVA, c: DEX, d: 50 mg/kg e: 125 mg/kg f: 250 mg/kg). As shown therein, the number of WBC cells significantly increased in the OVA-sensitized group compared to the control group, and dose-dependently decreased in the groups administered with various doses of oleic acid.

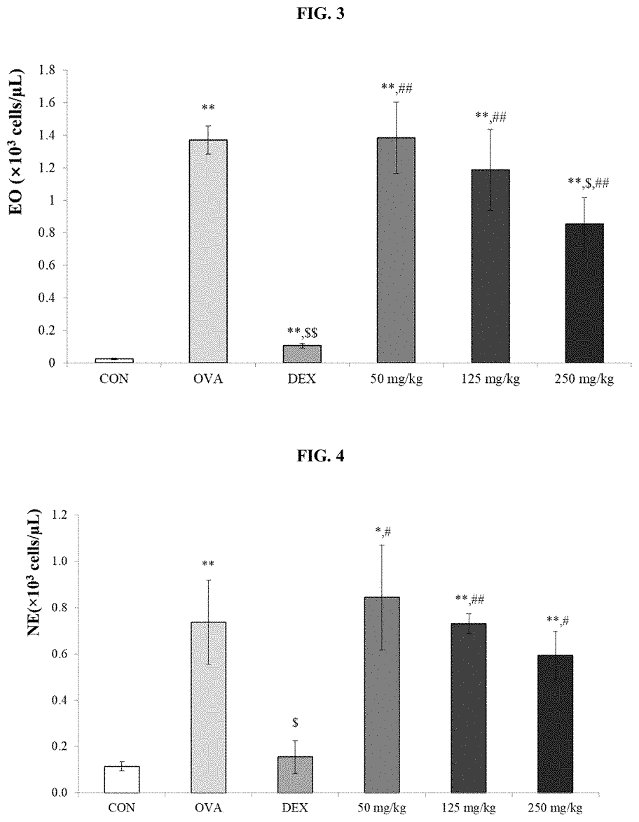

FIG. 3 shows the change in the number of eosinophils (EOs) in the OVA-sensitized group by administration of oleic acid (a: CON, b: OVA, c: DEX, d: 50 mg/kg, e: 125 mg/kg, f: 250 mg/kg). As shown therein, the number of EOs increased in the OVA-sensitized group compared to the control group, and dose-dependently decreased in the groups administered with various doses of oleic acid.

FIG. 4 shows the change in the number of neutrophils (NEs) in the OVA-sensitized group by administration of oleic acid (a: CON, b: OVA, c: DEX, d: 50 mg/kg, e: 125 mg/kg, f: 250 mg/kg). As shown therein, the number of NEs significantly increased in the OVA-sensitized group compared to the control group, and dose-dependently decreased in the groups administered with various doses of oleic acid.

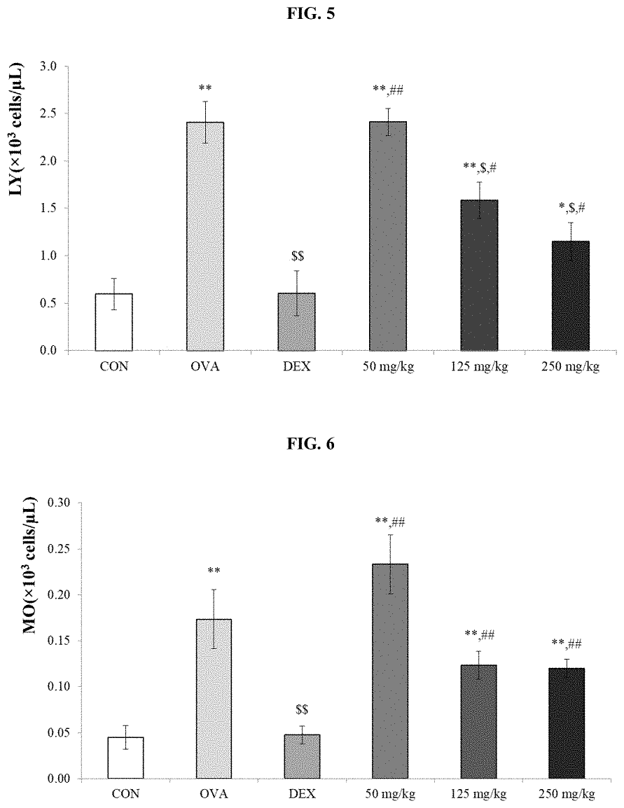

FIG. 5 shows the change in the number of lymphocytes (LYs) in the OVA-sensitized group by administration of oleic acid (a: CON, b: OVA, c: DEX, d: 50 mg/kg, e: 125 mg/kg, f: 250 mg/kg). As shown therein, the number of LYs significantly increased in the OVA-sensitized group compared to the control group, and dose-dependently decreased in the groups administered with various doses of oleic acid.

FIG. 6 shows the change in the number of monocytes (MOs) in the OVA-sensitized group by administration of oleic acid (a: CON, b: OVA, c: DEX, d: 50 mg/kg, e: 125 mg/kg, f: 250 mg/kg). As shown therein, the number of MOs significantly increased in the OVA-sensitized group compared to the control group, and dose-dependently decreased in the groups administered with various doses of oleic acid.

### (3) Results of BALF and Serum Analysis in Groups Administered with Palmitic Acid

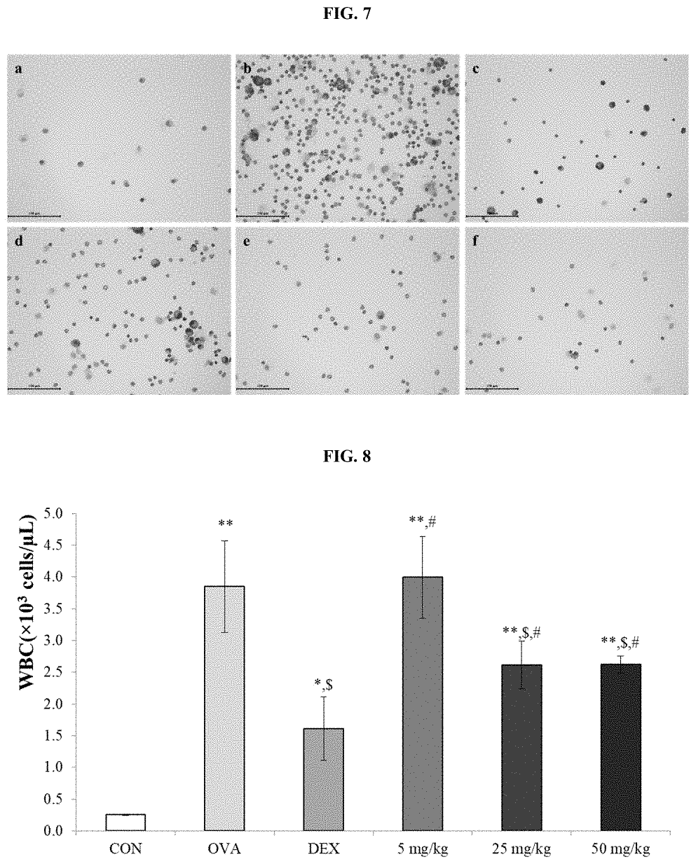

FIG. 7 shows the results measuring a differential cell count by a blood cell analysis in the BALF of the groups administered with palmitic acid (a: CON, b: OVA, c: DEX, d: 5 mg/kg, e: 25 mg/kg, f: 50 mg/kg). As can be seen therein, the number of differential cells dose-dependently decreased in the groups administered with palmitic acid.

FIG. 8 shows the change in the number of WBC cells in the OVA-sensitized group by administration of palmitic acid (a: CON b: OVA, c: DEX, d: 5 mg/kg, e: 25 mg/kg, f: 50 mg/kg). As shown therein, the number of WBC cells significantly increased in the OVA-sensitized group compared to the control group, and dose-dependently decreased in the groups administered with various doses of palmitic acid.

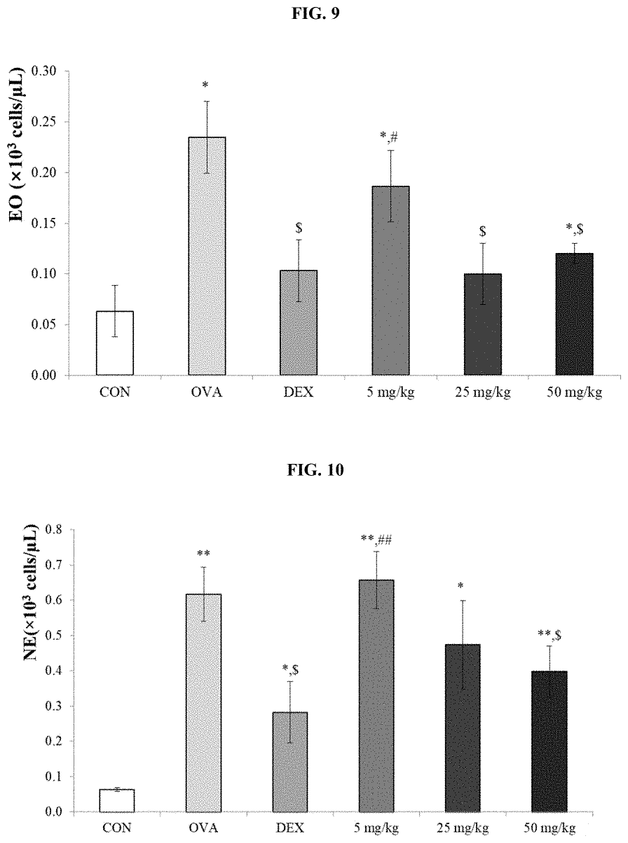

FIG. 9 shows the change in the number of eosinophils (EOs) in the OVA-sensitized group by administration of palmitic acid (a: CON, b: OVA, c: DEX, d: 5 mg/kg, e: 25 mg/kg, f: 50 mg/kg). As shown therein, the number of EOs increased in the OVA-sensitized group compared to the control group, and was the smallest in the groups administered with palmitic acid, particularly the group administered with 25 mg/kg of palmitic acid.

FIG. 10 shows the change in the number of neutrophils (NEs) in the OVA-sensitized group by administration of palmitic acid (a: CON, b: OVA, c: DEX, d: 5 mg/kg, e: 25 mg/kg, f: 50 mg/kg). As shown therein, the number of NEs significantly increased in the OVA-sensitized group compared to the control group, and dose-dependently decreased in the groups administered with various doses of palmitic acid.

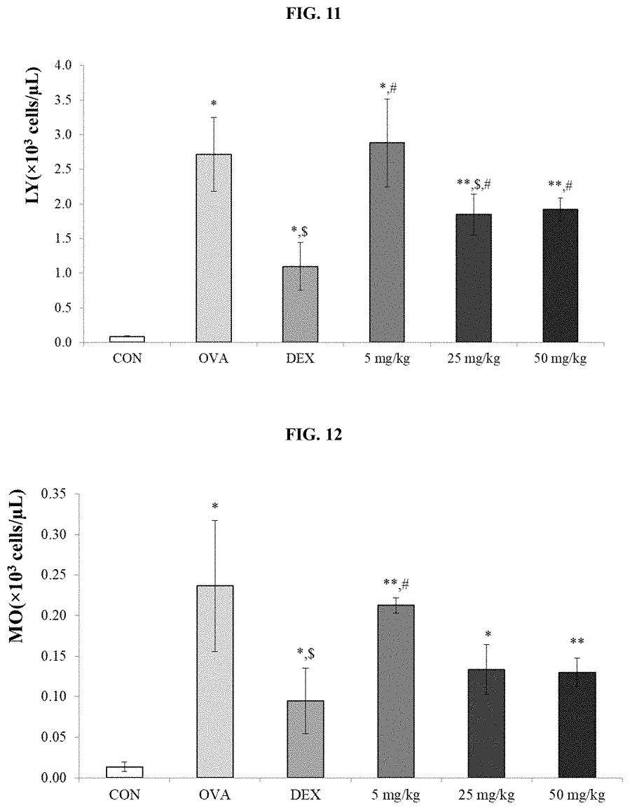

FIG. 11 shows the change in the number of lymphocytes (LYs) in the OVA-sensitized group by administration of palmitic acid (a: CON, b: OVA, c: DEX, d: 5 mg/kg, e: 25 mg/kg, f: 50 mg/kg). As shown therein, the number of LYs increased in the OVA-sensitized group compared to the control group, and was the smallest in the groups administered with palmitic acid, particularly the group administered with 25 mg/kg of palmitic acid.

FIG. 12 shows the change in the number of monocytes (MOs) in the OVA-sensitized group by administration of palmitic acid (a: CON, b: OVA, c: DEX, d: 5 mg/kg, e: 25 mg/kg, f: 50 mg/kg). As shown therein, the number of MOs significantly increased in the OVA-sensitized group compared to the control group, and dose-dependently decreased in the groups administered with various doses of palmitic acid.

### (4) Analysis of IgE in BALF

Immunoglobulin E (IgE) is a type of protein called antibody that plays an important role in allergic reactions. When IgE is exposed to a certain substance, the immune system begins to produce IgE in order to protect the body. As a result, when allergic asthma is induced, a large amount of IgE is produced in the body. The BALF samples obtained in the animal experiment were used to measure how IgE was expressed.

A BD Mouse IgE ELISA set (BD OptEIA, Cat No. 555248) was used, and reagents were prepared according to the data sheet of the kit. 100 μL of a capture antibody diluted in coating buffer was added to each coated micro-well, covered with a cover, and left to stand overnight 4° C. Each well was washed three times with 300 μL of washing buffer each time. 200 μL of an assay diluent was added to each well which was then incubated at room temperature for 1 hour. Each well was washed three times with 300 μL of washing buffer each time. 100 μL of each of a standard substance and sample diluted with an assay diluent was added to each well which was then incubated at room temperature for 2 hours. Each well was washed five times with 300 μL of washing buffer each time. 100 μL of a working detector (detection antibody+SAv-HRP reagent) was added to each well which was then incubated at room temperature for 1 hour. Each well was washed seven times with 300 μL of washing buffer each time, and then 100 μL of a TMB substrate solution was added to each well which was then incubated in the dark at room temperature for 30 minutes. 50 μL of a stop solution was added to each well, and the absorbance at 450 nm was measured.

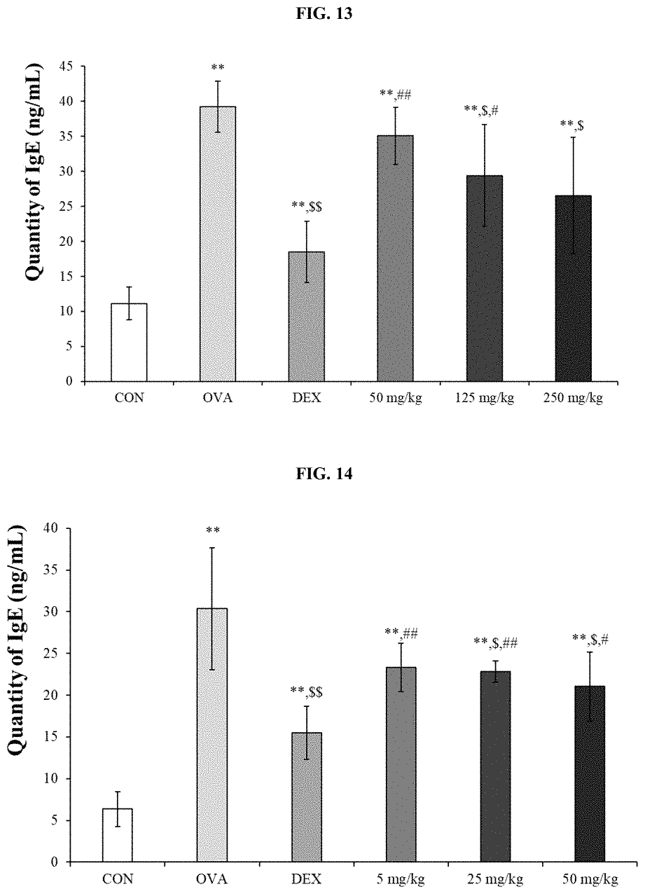

FIG. 13 shows the change in the quantity of IgE in the OVA-sensitized group by administration of oleic acid (a: CON, b: OVA, c: DEX, d: 50 mg/kg, e: 125 mg/kg, f: 250 mg/kg). As shown therein, the quantity of IgE significantly increased in the OVA-sensitized group compared to the control group, and dose-dependently decreased in the groups administered with various doses of oleic acid. FIG. 14 shows the change in the quantity of IgE in the OVA-sensitized group by administration of palmitic acid (a: CON, b: OVA, c: DEX, d: 5 mg/kg, e: 25 mg/kg, f: 50 mg/kg). As shown therein, the quantity of IgE significantly increased in the OVA-sensitized group compared to the control group, and dose-dependently decreased in the groups administered with various doses of palmitic acid.

### (5) H&E Staining

Hematoxylin & Eosin (H&E) is a staining method which is mostly widely used for tissue staining. Hematoxylin is basic and is used to stain phosphate-rich nuclei in tissue. Eosin is acidic, gives color by binding to a basic atomic group, and is used to stain outer cellular structures, cytoplasm, cell walls and the like.

Deparaffinization and hydration procedures were performed in xylene for 15 minutes, 80% alcohol for 1 minute, 90% alcohol for 1 minute, and 100% alcohol. After dehydration, the tissue was washed with single distilled water for 5 minutes, and then stained with heamatoxylin for 1 minute and 30 seconds. After staining, the tissue was washed with single distilled water for 15 minutes until it appeared blue. Clearing and dehydration were performed in in 95% alcohol for 1 minute, eosin for 6 minutes, 95% alcohol for 1 minute, 95% alcohol for 1 minute, 100% alcohol for 1 minute, and xylene for 6 minute, and then the tissue was mounted. After tissue mounting, microscopic observation was performed.

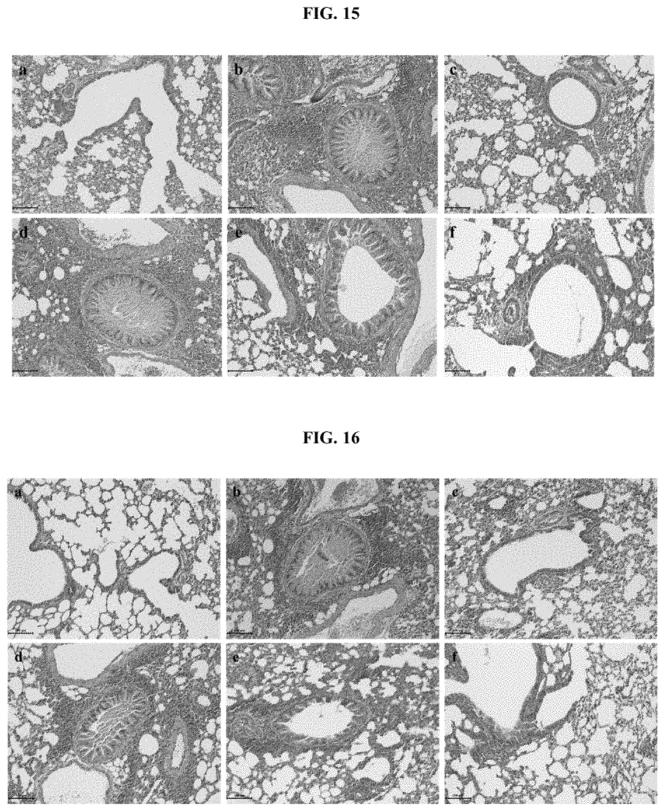

FIG. 15 shows the results of H&E staining for the OVA-sensitized group and the groups administered with oleic acid (a: CON, b: OVA, c: DEX, d: 50 mg/kg, e: 125 mg/kg, f: 250 mg/kg). As shown therein, eosinophils, mucus and epithelial hyperplasia increased in the OVA-sensitized group compared to the control group, and dose-dependently decreased in the groups administered with various doses of oleic acid.

FIG. 16 shows the results of H&E staining for the OVA-sensitized group and the groups administered with palmitic acid (a: CON, b: OVA, c: DEX, d: 5 mg/kg, e: 25 mg/kg, f: 50 mg/kg). As shown therein, eosinophils, mucus and epithelial hyperplasia increased in the OVA-sensitized group compared to the control group, and dose-dependently decreased in the groups administered with various doses of palmitic acid.

### (6) PAS Staining

Tissue contains polysaccharides, mucopolysaccharides, muco proteins, glycoproteins, etc. According to the principle of PAS staining, a carbohydrate group bound to these proteins is oxidized by periodic acid to produce an aldehyde group which then reacts with Schiff's reagent to give red purple.

Deparaffinization and hydration procedures were performed in xylene for 15 minutes, 80% alcohol for 1 minute, 90% alcohol for 1 minute, and 100% alcohol for 1 minute. Then, the tissue was oxidized by 0.5% periodic acid solution for 10 minutes. After oxidation, the tissue was washed with running tap water for 5 minutes and washed lightly with single distilled water. After washing, the tissue was reacted with Schiff reagent for 10 minutes, and then washed with running tap water for 5 minutes. After washing, the tissue was stained with Hematoxylin for 1 minute and 30 second, and washed again with running water for 10 minutes. After washing, the tissue was mounted, followed by microscopic observation.

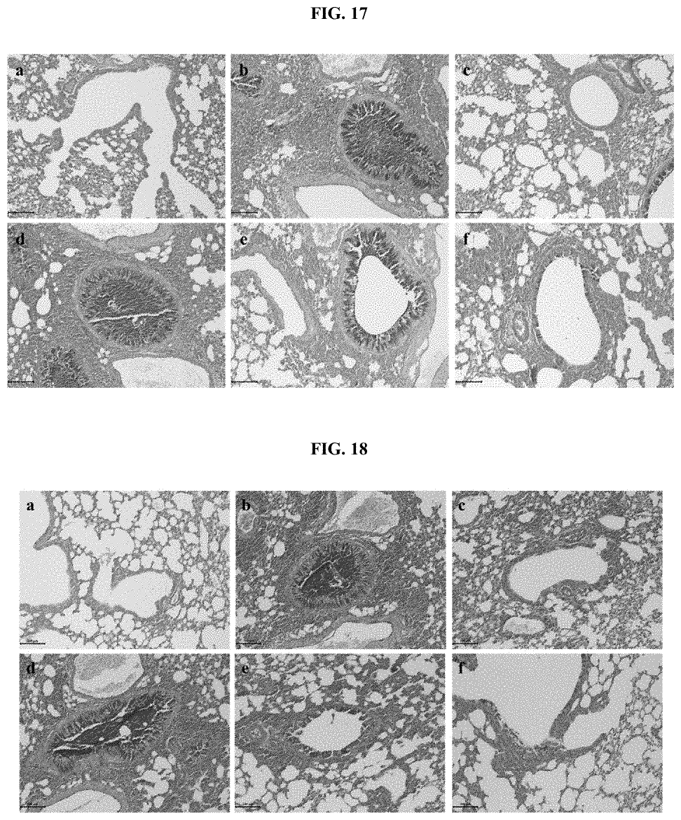

FIG. 17 shows the results of PAS staining for the OVA-sensitized group and the groups administered with oleic acid (a: CON, b: OVA, c: DEX, d: 50 mg/kg, e: 125 mg/kg, f: 250 mg/kg). As shown therein, mucus increased in the OVA-sensitized group compared to the control group, and dose-dependently decreased in the groups administered with various doses of oleic acid.

FIG. 18 shows the results of PAS staining for the OVA-sensitized group and the groups administered with palmitic acid (a: CON, b: OVA, c: DEX, d: 5 mg/kg, e: 25 mg/kg, f: 50 mg/kg). As shown therein, mucus increased in the OVA-sensitized group compared to the control group, and dose-dependently decreased in the groups administered with various doses of palmitic acid.

### (7) Immunofluorescent Analysis

In order to find the localizations of the Th1 cell transcription factor T-bet and the Th2 cell transcription factor GATA-3, immunofluorescent analysis was performed, and four groups were measured: a control group, an OVA-sensitized group, a group treated with dexamethasone after asthma induction by ovalbumin, and a group treated with 1500 mg/kg macmoondong-tang after asthma induction by ovalbumin.

Prior to the antibody binding step, the same materials for immunohistochemical analysis but rabbit anti-mouse T-bet (Biorbyt, orb7075, Cambridge, UK) or goat anti-mouse GATA-3 (OriGene, TA305795, Rockville, Md., USA) were used as primary antibodies for 1 hour at room temperature. The slides were incubated for 2 hours with FITC-conjugated anti-rabbit IgG (Jackson Immunoresearch, 315-095-003, West Grove, Pa., USA) or Alexa Flur 555-conjugated anti-goat IgG (ThermoFisher Scientific, A-21127, Waltham, Mass., USA) and the cells were counterstained with DAPI (ThermoFisher Scientific, 62249, Waltham, Mass., USA). The images were obtained using a K1-Fluo confocal microscope (Nanoscope System, Daejeon, South Korea).

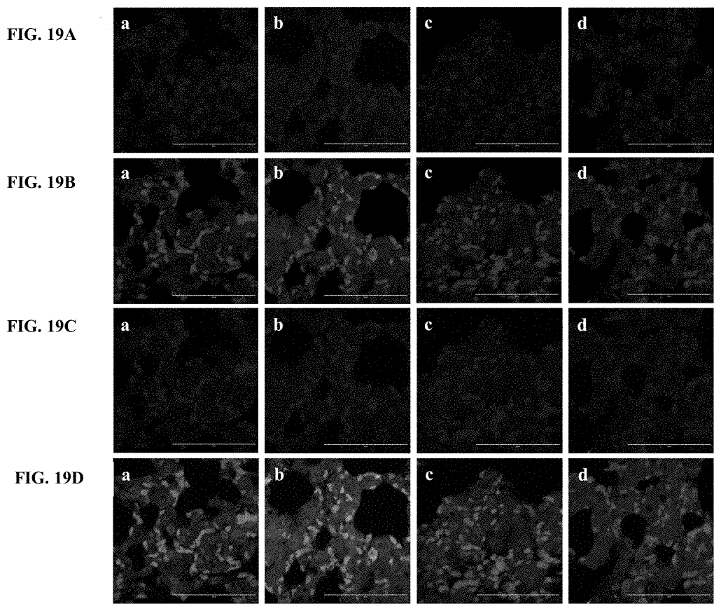

FIG. 19A-19D shows the changes in activities of the Th1 cell transcription factor T-bet and the Th2 cell transcription factor GATA-3 by administration of palmitic acid. As can be seen therein, in the asthma-induced group (b), T-bet and GATA-3 were all activated in the nucleus, indicating that the functions of the transcription factors were activated.

### (8) Enzyme-Linked Immunoassay (ELISA) Analysis

To analyze the levels of IFN-γ, IL-12p40, IL-4, IL-5, IL-6, and TNF-α in lung tissue, OptEIA mouse ELISAs were purchased from BD Biosciences. The IL-13 levels were assessed using the AbFrontier Cymax mouse FT ISA kit (AbFrontier, Seoul, Korea). All assays were performed according to the manufacturers' guidelines. All lung samples were prepared by a lysis buffer made with a protease inhibitor cocktail and an RIPA buffer (Thermo Fisher Scientific). Aliquots of lung tissue from all groups were weighed and homogenized with lysis buffer. They were then centrifuged at 8,200 rpm for 15 minutes, and the supernatants were harvested and measured using a microplate reader (EZ Read 400, Biochrom, Cambourne, UK).

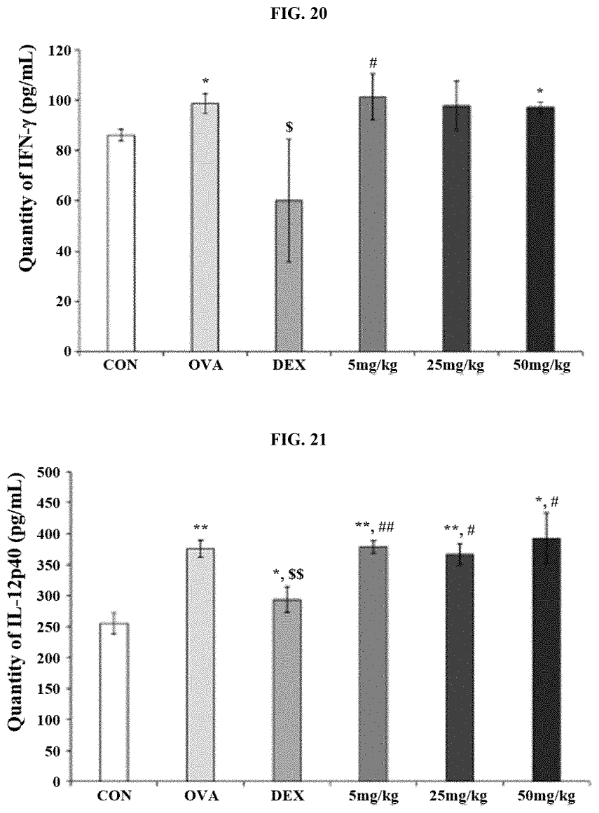

FIG. 20 shows the change in expression level of IFN-γ in the OVA-sensitized group by administration of palmitic acid. As shown therein, the expression level of IFN-γ increased in OVA compared to CON, and dose-dependently decreased in the groups administered with various doses of palmitic acid.

FIG. 21 shows the change in expression level of IL-12p40 in the OVA-sensitized group by administration of palmitic acid. As shown therein, the expression level of IL-12p40 increased in OVA compared to CON, and decreased in the groups administered with palmitic acid, particularly the group administered with 25 mg/kg of palmitic acid.

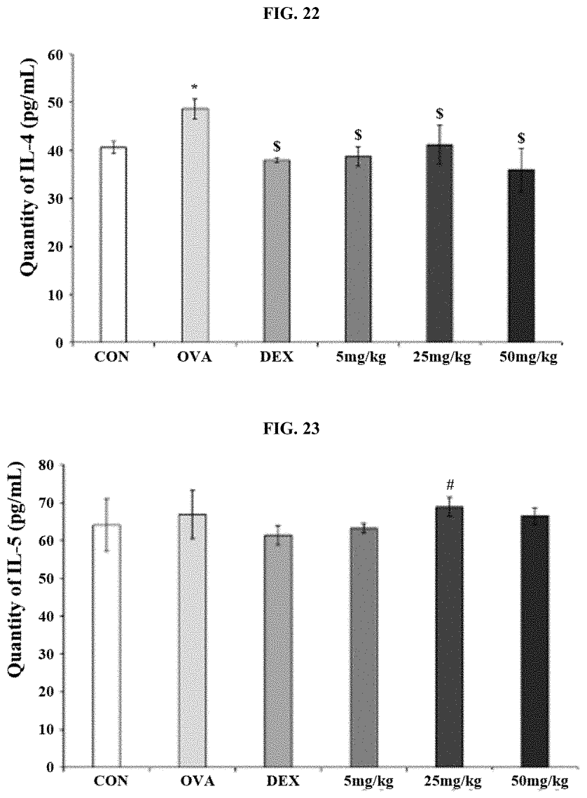

FIG. 22 shows the change in expression level of IL-4 in the OVA-sensitized group by administration of palmitic acid. As shown therein, the expression level of IL-4 increased in OVA compared to CON, and decreased in the groups administered with palmitic acid, particularly the group administered with 50 mg/kg of palmitic acid.

FIG. 23 shows the change in expression level of IL-5 in the OVA-sensitized group by administration of palmitic acid. As shown therein, the expression level of IL-5 increased in OVA compared to CON, and decreased in the groups administered with palmitic acid, particularly the group administered with 5 mg/kg of palmitic acid.

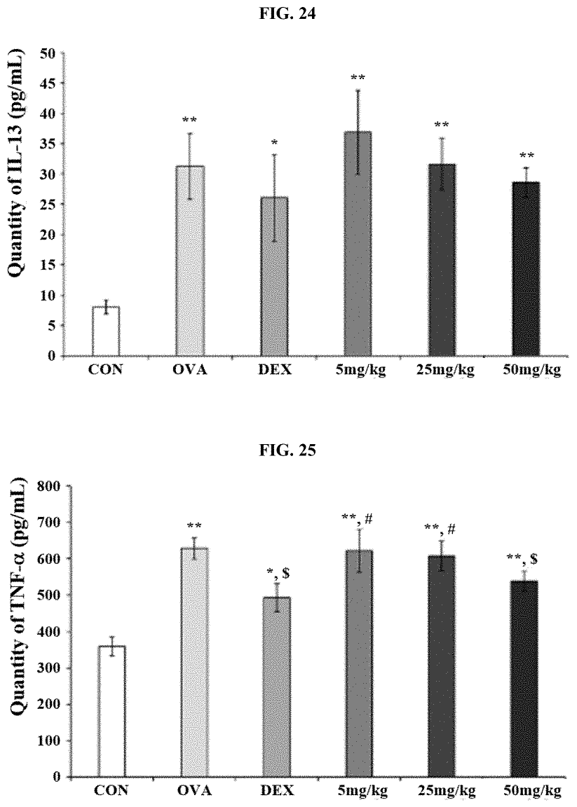

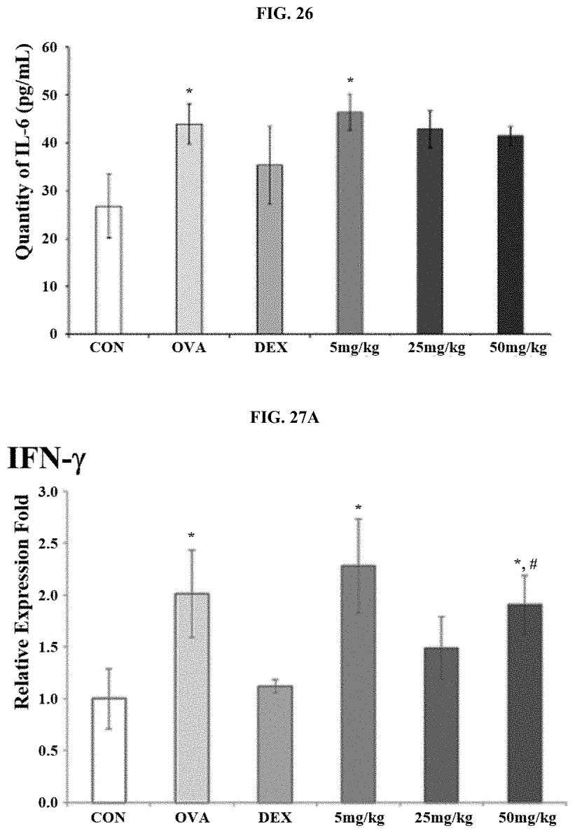

FIG. 24 shows the change in expression level of IL-13 in the OVA-sensitized group by administration of palmitic acid. As shown therein, the expression level of IL-13 increased in OVA compared to CON, and decreased in the groups administered with palmitic acid, particularly the group administered with 50 mg/kg of palmitic acid.

FIG. 25 shows the change in expression level of TNF-α in the OVA-sensitized group by administration of palmitic acid. As shown therein, the expression level of TNF-α increased in OVA compared to CON, and decreased in the groups administered with palmitic acid, particularly the group administered with 50 mg/kg of palmitic acid.

FIG. 24 shows the change in expression level of IL-6 in the OVA-sensitized group by administration of palmitic acid. As shown therein, the expression level of IL-6 increased in OVA compared to CON, and decreased in the groups administered with palmitic acid, particularly the group administered with 50 mg/kg of palmitic acid.

### (9) Reverse Transcription-Polymer Chain Reaction (RT-PCR) Analysis

In order to evaluate changes in the cDNA levels of IFN-γ, IL-12p40, IL-4, IL-5, IL-13, TNF-α, and IL-6, which are related to asthma induction, RT-PCR analysis was performed as in a previous study protocol (Lee et al., 2017). Total RNA was extracted from the lung using the RNeasy Mini Kit (Qiagen, Hilden, Germany) according to the manufacturer's instructions. Total RNA (100 ng) was used as a template for the reaction. Primers for RT-PCR were synthesized as shown in Table 1 below.

TABLE 1

| | | | |
|---|---|---|---|
| IFN-γ | Forward | 5′-GGCCATCAGCAA | SEQ ID |
| | | CAACATAAG-3′ | NO: 1 |
| | Reverse | 5′-GTTGACCTCAAA | SEQ ID |
| | | CTTGGCAATAC-3′ | NO: 2 |
| IL-12p40 | Forward | 5′-GGACCAAAGGGA | SEQ ID |
| | | CTATGAGAAG-3′ | NO: 3 |
| | Reverse | 5′-CTTCCAACGCCA | SEQ ID |
| | | GTTCAATG-3′ | NO: 4 |
| IL-5 | Forward | 5′-TGCATCAGGGTC | SEQ ID |
| | | TCAAGTATTC-3′ | NO: 5 |
| | Reverse | 5′-GGATGCTAAGGT | SEQ ID |
| | | TGGGTATGT-3′ | NO: 6 |
| IL-13 | Forward | 5′-CAGCCCTCAGCC | SEQ ID |
| | | ATGAAATA-3′ | NO: 7 |
| | Reverse | 5′-CTTGAGTGTGTA | SEQ ID |
| | | ACAGGCCATTCT-3 | NO: 8 |
| IL-6 | Forward | 5′-GATAAGCTGGAG | SEQ ID |
| | | TCACAGAAGG-3′ | NO: 9 |
| | Reverse | 5′-TTGCCGAGTAGA | SEQ ID |
| | | TCTCAAAGTG-3′ | NO: 10 |
| TNF-α | Forward | 5′-CTGAGTTCTGCA | SEQ ID |
| | | AAGGGAGAG-3′ | NO: 11 |
| | Reverse | 5′-CCTCAGGGAAGA | SEQ ID |
| | | ATCTGGAAAG-3′ | NO: 12 |
| GAPDH | Forward | 5′-GTGGAGTCATAC | SEQ ID |
| | | TGAACATGTAG-3′ | NO: 13 |
| | Reverse | 5′-AATGGTGAAGGT | SEQ ID |
| | | CGGTGTG-3′ | NO: 14 |

The RT-PCR cycles consisted of denaturation at 95° C. for 5 sec and annealing/extension at 65° C. for 30 sec for 40 cycles. The results were obtained using an Axioscope A1 (Carl Zeiss, Gottingen, Germany).

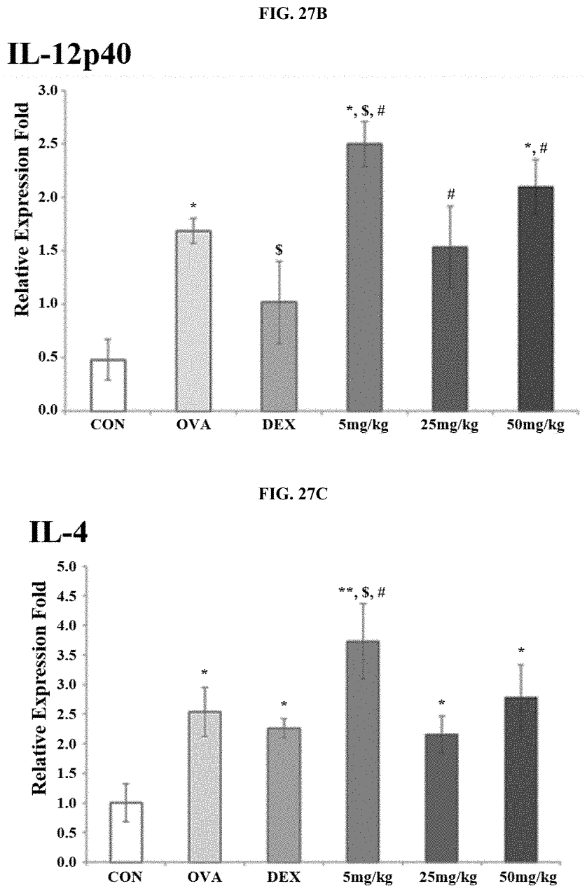

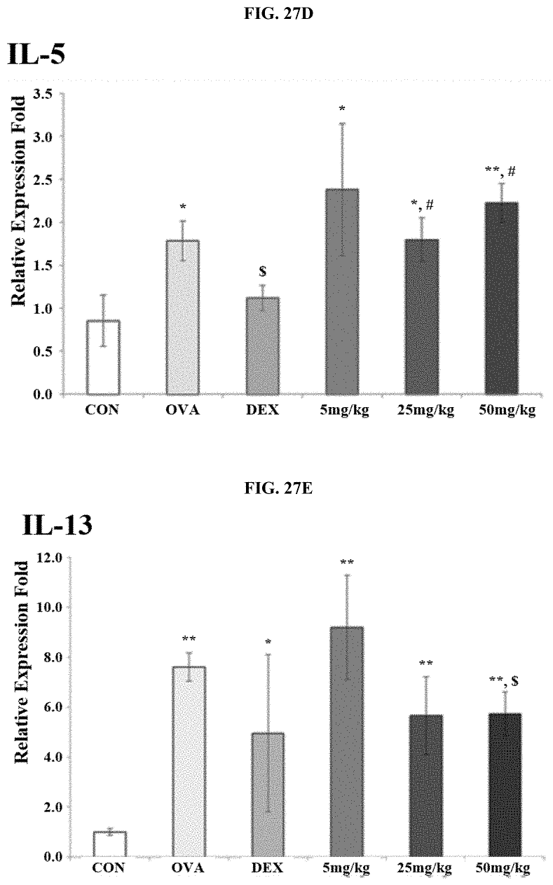

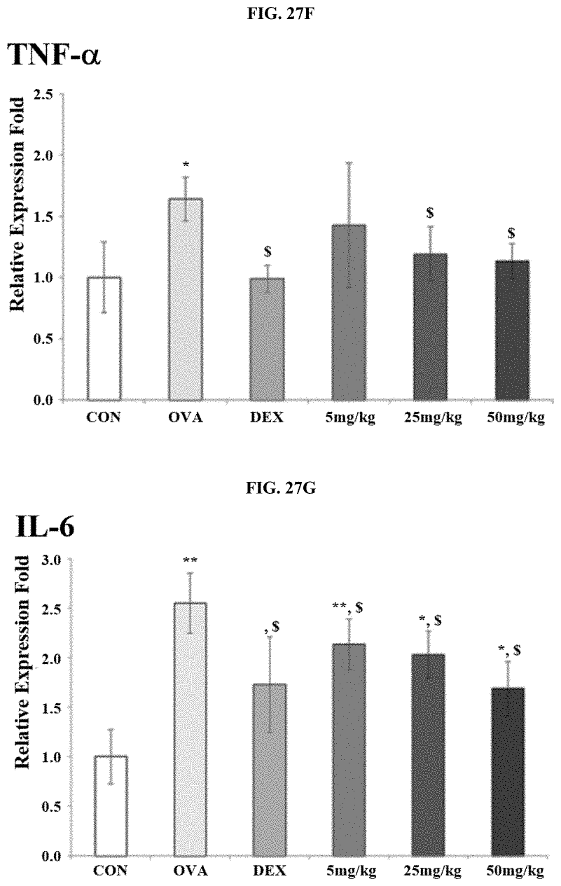

FIG. 27 shows the changes in helper T cell-related cytokines by administration of palmitic acid. As shown therein, the Th2-related cytokine IL-13 and the Th17-related cytokines TNF-α and IL-6 dose-dependently decreased by administration of palmitic acid.

As described above, the saturated or unsaturated fatty acid having 12 to 22 carbon atoms according to the present invention functioned to inhibit or eliminate various asthma symptoms induced by ovalbumin. Specifically, it could be seen that the fatty acid inhibited the proliferation of leukocytes, neutrophils, lymphocytes and monocytes in BALF and serum, inhibited the proliferation of bronchial epithelial cells, reduced mucus in bronchioles, and reduced infiltration of many inflammatory cells around bronchioles and blood vessels in a dose-dependent manner. In addition, the fatty acid induced a decrease in the expression of IFN-γ, IL-12p40, IL-4, IL-5, IL-13 and TNF-α, which are Th2 cell-related cytokines. Therefore, the saturated or unsaturated fatty acid having 12 to 22 carbon atoms according to the present invention is a compound which overcomes the side effects of currently used asthma therapeutic agents, has an excellent therapeutic effect without having toxicity, and may advantageously be used as a composition for preventing, treating or alleviating asthma.

1. A method for treating asthma, comprising:
administering a composition comprising an unsaturated fatty acid having 12 to 22 carbon atoms or a pharmaceutically acceptable salt thereof in an amount of 0.1 to 10 wt % based on the total weight of the composition, as an active ingredient, to a subject in need thereof.

2. The method of claim 1, wherein the saturated fatty acid is selected from the group consisting of lauric acid, myristic acid, palmitic acid, stearic acid and a mixture thereof.

3. The method of claim 1, wherein the unsaturated fatty acid is selected from the group consisting of oleic acid, linoleic acid, linolenic acid and a mixture thereof.

4. The method of claim 1, wherein the composition is a food composition or a pharmaceutical composition.


SEQUENCE LISTING (14 sequences)

SEQ ID NO: 1 - DNA, 21 bp, Artificial Sequence
  IFN-gama forward primer
ggccatcagcaacaacataag

SEQ ID NO: 2 - DNA, 23 bp, Artificial Sequence
  IFN-gama reverse primer
gttgacctcaaacttggcaatac

SEQ ID NO: 3 - DNA, 22 bp, Artificial Sequence
  IL-12p40 forward primer
ggaccaaagggactatgagaag

SEQ ID NO: 4 - DNA, 20 bp, Artificial Sequence
  IL-12p40 reverse primer
cttccaacgccagttcaatg

SEQ ID NO: 5 - DNA, 22 bp, Artificial Sequence
  IL-5 forward primer
tgcatcagggtctcaagtattc

SEQ ID NO: 6 - DNA, 21 bp, Artificial Sequence
  IL-5 reverse primer
ggatgctaaggttgggtatgt

SEQ ID NO: 7 - DNA, 20 bp, Artificial Sequence
  IL-13 forward primer
cagccctcagccatgaaata

SEQ ID NO: 8 - DNA, 24 bp, Artificial Sequence
  IL-13 reverse primer
cttgagtgtgtaacaggccattct

SEQ ID NO: 9 - DNA, 22 bp, Artificial Sequence
  IL-6 forward primer
gataagctggagtcacagaagg

SEQ ID NO: 10 - DNA, 22 bp, Artificial Sequence
  IL-6 reverse primer
ttgccgagtagatctcaaagtg

SEQ ID NO: 11 - DNA, 21 bp, Artificial Sequence
  TNF-alpha forward primer
ctgagttctgcaaagggagag

SEQ ID NO: 12 - DNA, 22 bp, Artificial Sequence
  TNF-alpha reverse primer
cctcagggaagaatctggaaag

SEQ ID NO: 13 - DNA, 23 bp, Artificial Sequence
  GAPDH forward primer
gtggagtcatactgaacatgtag

SEQ ID NO: 14 - DNA, 19 bp, Artificial Sequence
  GAPDH reverse primer
aatggtgaaggtcggtgtg